(19) United States
(12) Patent Application Publication
(10) Pub. No.: US 2003/0109817 A1
(43) Pub. Date: Jun. 12, 2003

(54) Supplementary knee support brace

(76) Inventor: Shimon Berl, Kiryar Bialik (IL)

Correspondence Address: WELSH & FLAXMAN LLC, 2341 JEFFERSON DAVIS HIGHWAY, SUITE 112, ARLINGTON, VA, 22202, US

(21) Appl. No.: 10/011,407

(22) Filed: Dec. 11, 2001

(51) Int. Cl. A61F 5/00

(52) U.S. Cl. 602/5

(57) ABSTRACT

The present invention is a device that provides supplementary support to a human knee in such a way as to unload pressure from the knee joint, while standing or walking. This is accomplished by transferring a portion of the load through the device of the present invention and either back onto the leg, onto the surface upon which the leg is resting, or a combination of both. The present invention is able to accommodate changes in the distance over which the load is transferred while maintaining the tension necessary for effective load transfer. This coupled with different embodiments of hinge elements that offer multi-degrees of freedom give the present invention the ability to accommodate misalignment of the hinge with the axis of rotation of the knee a allow for a more natural rotation of the knee. Additionally, the system and teachings of the present invention may be adapted for use in conjunction with joints other than the knee or even for use in veterinary applications.

### FIELD AND BACKGROUND OF THE INVENTION

The present invention relates to a knee brace and, in particular, it concerns a supplementary support brace for a human leg that significantly reduces the load applied to the knee by a predetermined magnitude during the course of a step or while standing.

There are known knee braces that provide some support for the knee. These range from elastic wrap-braces with no rigid components to braces that have rigid frames. Some of the braces with rigid frames include frame members that contact the surface on which the foot is resting. Many braces are effective in protecting the knee from undesirable side forces and movement.

One method used to take pressure off the damaged portion of the knee is to apply lateral pressure to the knee or the leg close to the knee. This lateral pressure forces a shift in the angle at which the bones of the leg come together at the knee. While this may relieve pressure for one area, it adds pressure to other areas, thereby increasing the rate at which those areas are worn. It is also not effective in cases where the knee suffers from bi-compartmental or tri-compartmental damage.

The orthopedic brace of U.S. Pat. No. 6,010,474 to Wycoki is representative of the braces that unload the knee by using devices that attach to the leg above the knee and use a rigid frame to transfer the load to the ground. Braces of this style by-pass the soft tissue below the knee and transfer load directly to the ground. In this group, too, the problems of the component shifting on the soft tissue above the knee and the use of fixed hinges that do not rotate with the same degrees of freedom as a human knee joint still exist. It should be noted that devices based on the Wycoki braces are not in common use.

A common problem is the lack of a solid anchor point for load transference. Braces that are attached to the leg above and below the knee are anchored to substantially vertical body parts by substantially vertical anchoring components. This situation is exacerbated by the characteristics of soft tissue. This may result in the components of the brace shifting position on the leg, thereby loosing some magnitude of load transference.

There is therefore a need for a supplementary support brace for a human leg that significantly reduces the load applied to the knee, while standing or walking, and allows rotation commensurate with the natural bending of the knee.

### SUMMARY OF THE INVENTION

The present invention is a device that provides supplementary support to a human leg that reduces the load applied to the knee while walking or standing.

According to the teachings of the present invention there is provided, 1. A device that provides supplementary support to a joint so as to reduce the load applied to the joint by transferring a portion of the load through the device, the joint being located between a proximal body portion and a distal body portion, the device comprising: (a) a proximal support system including at least one proximal support element, and at least one proximal attachment structure, the proximal attachment structure configured for attachment to portions of the proximal body portion and for transfer of the portion of the load between the proximal body portion and the proximal support system; (b) a distal support system including at least one distal support element, and at least one distal attachment structure, the distal attachment structure configured for attachment to portions of the distal body portion and for transfer of the portion of the load between the distal body portion and the distal support system; and (c) a load-transfer system interconnected with the proximal support system and the distal support system, the load-transfer system including: (i) a load-distance compensator configured so as to accommodate changes in the distance over which the portion of the load is transferred while supplying a force so as to cause the proximal support system and the distal support system to move substantially away from each other; associated with (ii) at least one hinged element configured so as to allow rotation of the proximal and distal support systems in relation to each other so that the rotation accommodates natural bending of the joint.

According to a further feature of the present invention, there is also provided a control mechanism, the control mechanism selectively regulating the load-distance compensation device. Optionally, a trigger mechanism is configured to actuate the control mechanism.

According to a further feature of the present invention, the load-distance compensator supplies a substantially constant force.

According to a further feature of the present invention, the load-distance compensator includes a mechanical spring mechanism.

According to a further feature of the present invention, the load-distance compensator includes a pneumatic spring mechanism.

According to a further feature of the present invention, the load-distance compensator includes a combination of spring mechanisms.

According to a further feature of the present invention, the hinge element includes a member with one degree of translational freedom of movement, the movement being substantially perpendicular to a direction of the force.

According to a further feature of the present invention, the hinge element includes a bracket associated with a first of the proximal and distal support elements, and further includes at least one pin associated with a second of the proximal and distal support systems, the bracket containing at least one elongated slot, the elongation being in a direction substantially perpendicular to the first support element and substantially parallel to a plane that is perpendicular to a primary axis of rotation of the knee, the pin being fitted into the slot, the pin being substantially parallel to the primary axis of rotation of the knee, the slot allowing for sliding movement and rotation of the pin.

According to a further feature of the present invention, the hinge element includes a leaf spring fixedly attached to corresponding ends of the proximal support element and the distal support element, the spring configured so as to bend in a direction compatible with a primary direction of rotation of the joint, the spring being encased by a plurality of bend restriction links, the links configured so as to allow the spring to bend within rotational limits of the joint while restricting the bending outside of the rotational limits of the joint.

There is also provided according to the teachings of the present invention, a device that provides supplementary support to a human leg so as to reduce the load applied to the knee while standing or walking by transferring a portion of the load through the device, the device comprising: (a) an upper support system including at least one upper support element, and at least one upper attachment structure, the upper attachment structure configured for attachment around portions of the leg above the knee and for transfer of the portion of the load between the leg above the knee and the upper support system; (b) a lower support system including at least one lower support element, and at least one lower attachment structure, the lower attachment structure configured for attachment around portions of the leg below the knee and transfer of the portion of the load between the leg below the knee and the lower support system; and (c) a load-transfer system interconnected with the upper support system and the lower support system, the load-transfer system including: (i) a load-distance compensator configured so as to accommodate changes in the distance over which the portion of the load is transferred while supplying a force so as to cause the upper support system and the lower support system to move substantially away from each other; associated with (ii) at least one hinged element configured so as to allow rotation of the upper and lower support systems in relation to each other so that the rotation accommodates bending of the knee.

According to a further feature of the present invention, the lower support system is supported by, and transfers the portion of the load solely to, the leg below the knee.

According to a further feature of the present invention, there is also provided a surface contact extension interconnected with the lower support element, the lower support element being supported by attachment to the leg and additionally supported by the surface contact extension coming in contact with a surface upon which a foot of the leg steps, the portion of the load being transferred to the leg and the surface.

According to a further feature of the present invention, the lower support element is supported primarily by the surface contact extension and the portion of the load is applied primarily to the surface, the lower support element being attached to the leg primarily for positioning purposes.

According to a further feature of the present invention, there is also provided a control mechanism, the control mechanism selectively restricting downward extension of the surface contact extension.

### BRIEF DESCRIPTION OF THE DRAWINGS

The invention is herein described, by way of example only, with reference to the accompanying drawings, wherein:

FIG. 1 is a partial cut-away side view of a preferred embodiment of the present invention, which includes a mechanical spring force supplying device and slotted multi-degree of freedom hinge element, attached to a leg;

FIGS. 2a and 2b show front and side elevations of a preferred embodiment of a slotted multi-degree of freedom hinge element of the present invention;

FIG. 3 is a cut-away side view and a cross-section of a preferred embodiment of a pneumatic spring force generating mechanism;

FIG. 4 is a series of side, front, top, and bottom views of a preferred embodiment of a leaf spring multi-degree of freedom hinge;

FIG. 5 is a cut-away side view of preferred embodiment of a hydraulic pressure actuated control mechanism of the present invention.

### DESCRIPTION OF THE PREFERRED EMBODIMENTS

The present invention is a supplementary support brace for a human leg that reduces the load applied to the knee while walking or standing.

The principles and operation of a supplementary support brace according to the present invention may be better understood with reference to the drawings and the accompanying description.

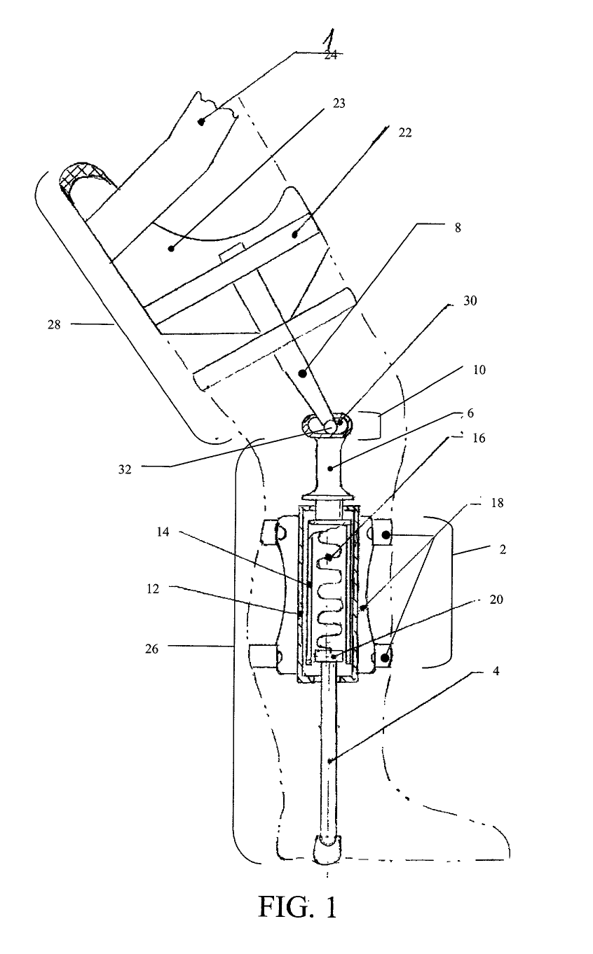

Referring now to the drawings, FIG. 1 shows one side of a preferred embodiment of the present invention attached to a human leg. This preferred embodiment includes corresponding device components deployed on the opposite side of the leg. However, since these components are not shown in the figures below, those shown will be referred to in the singular.

It is an intention of the present invention to significantly unload the knee of stress placed on it during the course of a step or while standing. This is accomplished by transferring the load above the knee to an external structure that then transfers part of the load either directly to the ground, back to the leg below the knee, or a combination of both. Although this concept of load transference is not new, the problem of the lack of solid anchor points due movement by the flexible soft tissue of the leg to which braces are attached greatly affects the load transference. The present invention overcomes this problem by using a load-distance compensator that compensates for shifting of device components

The load-distance compensator 2 provides force that causes the upper support system 28 and the lower support system 26 to move substantially away from each other. When in position, however, the movement of the upper and lower support systems away from each other is limited by the part of the leg around which they are attached. When in use, as the user takes a step, pressure is applied to the support systems of the device and the load is transferred to the device and off the knee joint. Due to movement during use, the positioning of the systems on the leg may shift, as in braces of prior art. This shifting may create problems with the amount of load transferred to the device. This is overcome by the load-distance compensator. The load-distance compensator maintains a substantially constant force of separation between the systems of the present invention while accommodating any changes in the distance across which the load is transferred. That is to say, if during use the upper support system were to shift to a position higher on the leg the distance along the leg over which load is to be transferred is longer. The spring element of the load-distance compensator would extent to compensate for the change in location of the upper support system and the greater distance between the new anchor point of the upper support system and the anchor point of the lower support system.

The embodiment shown here is configured with a load-distance compensator that uses a mechanical spring. The spring 16 transfers force elastically between piston 14 and contact extension 4 which are both displaceable longitudinally within the housing 12. As shown here, the piston is connected to the lower support element 6, and the housing is attached to the leg by attachment straps 18 which fasten around the leg. In this implementation, since both piston 14 and contact extension 4 are free to move within a predefined range vertically within housing 12, the attachment of the housing to the leg serves only to hold the device in position correctly aligned relative to the leg but is insulated from the vertical forces transferred by the device. The magnitude of the force the spring applied may be predetermined or adjustable. The upwardly directed force is transferred from the lower support element 6 to the upper support element 8 through the hinge element 10. It is preferable that the upper and lower support elements be configured to be primarily rigid while allowing lateral flexibility thereby providing for conformation with the shape of the respective portions of the leg to which they are attached. The downwardly directed force is applied to the ground by the surface contact extension 4. In the course of a step, the load is transferred to the upper support system 28, which is attached with straps 22 around the thigh and straps 24 around the lower pelvic region. The load is that transferred through the hinge element to the load-distance compensator. From the load-distance compensator the load is transferred to the ground contact extension and finally to the ground. Alternative implementations in which part or all of the forces are transferred to the lower leg below the knee will be discussed below.

The placement of the upper support system and straps may vary depending on the amount of load to be transferred. The support system and straps may be positioned around the thigh. In cases where a large amount of load transference is required, force transfer by fastening to the thigh is usually not sufficient due to the fact that the applied forces are substantially parallel to the surfaces of the thigh. In this case, the upper support system preferably includes a support structure 23 configured for deployment so as to transfer forces to a bone from which a more direct load transfer may occur. Specifically, use is made of a bone structure chosen to provide a force transfer interface significantly inclined relative to the direction of force transfer. In other words, the surface of the bones against which support structure 23 is positioned is chosen to be significantly non-parallel to the direction of force transfer, and preferably inclined thereto by at least about 30°, and more preferably by at least 45°. In such cases the load may be applied to a surface contact element and then directly to the ground. By way of non-limiting example, the support structure 23 may be configured to tuck up under the buttocks and transfer load from the lower portion of the pelvic bone through the systems of the present invention, including a surface contact extension, and directly to the surface upon which the foot is resting. In such a case, the straps may wrap around the thigh and the waist, or any other appropriate configuration. Clearly, the support structure may additionally, or alternatively, be configured to transfer forces via other bone structures in the lower pelvic region. Further suitable anatomic structures will be clear to one ordinarily skilled in the art, as discussions on this matter are common knowledge, U.S. Pat. No. 6,010,474 to Wycoki being one reference.

Since the leg bends at the knee during the process of taking a step, the direction in which the force of the load-distance compensator is applied will vary with the movement of the leg. The force may be supplied substantially constantly while the supplementary knee support brace is in place. Substantially constant being defined as variations in the force supplied of + or −20%, and preferably + or −10%. Alternatively, the force may be restricted as required. In such a case, a control mechanism 20 may be implemented.

An embodiment of a hinge element 10 of the present invention transfers load and force between the upper and lower support systems and provides rotation of the upper 28 and lower 26 support systems in relation to each other, while allowing the knee the freedom to move in a natural manner. The hinge element 10 shown here includes an elongated slot 30 that allows the pin 32 to rotate and slide within the slot. This hinge, coupled with the ability of the spring mechanism 16 of the load-distance compensator 2 to compensate for misalignment of the joint and the hinge, allows rotation with multiple degrees of freedom that better accommodates the natural rotation of a human knee. In some instances, the spring mechanism may also accommodate motion of the hinge to bring it into improved alignment with the knee.

It should be noted that while by example the load-distance compensator shown here is incorporated below the hinge, it might alternately be incorporated above the hinge. Further, the lower support system shown here includes a surface contact extension that transfers load to the surface of the ground. In certain circumstances, it may be advantageous to have portions of the load-distance compensator located above and below the hinge element. This may include the use of two load-distance compensators, one above and one below the knee.

As mentioned earlier, it is also within the intentions of the present invention to provide a lower support system configured to apply force solely to the leg below the knee. That is, a supplementary supports that transfers load from the leg above the knee to the leg below the knee. In this case, spring 16 acts directly on housing 12 so as to transfer forces via straps 18. The load may also be transferred back onto both the leg and the surface on which the foot rests. This is preferably achieved by providing a secondary spring (not shown) which provides a desired degree of force transfer to housing 12 while the remaining portion of the forces are transferred to surface contact extension 4. Furthermore, while the location of the hinge element shown in the examples of embodiments given here is at the junction of the ends of the upper and lower support elements, the location of the hinge may vary. Alternative locations for the hinge may include, but no be limited to, a junction at point a long the length of either or both of the support elements, or off set from the support elements, either to the front or the back, by brackets. These brackets may be, but not limited to “L” shaped brackets extending to the front or back. Additionally, the system and teachings of the present invention may be adapted for use in conjunction with joints other than the knee or even for use in veterinary applications.

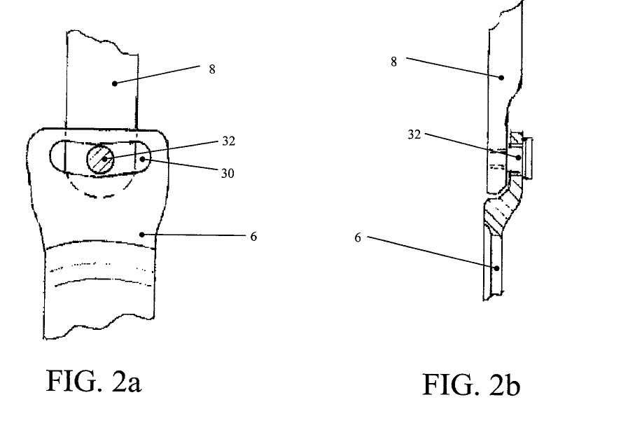

FIGS. 2a and 2b provide an example of a preferred embodiments of the slotted multi-degree of freedom hinge element mentioned in FIG. 1 above. It should be noted that while in the example discussed in FIGS. 2a and 2b the pin or pins are associated with the upper support element and the slot or slots are associated with the lower support element, this association is not an intention of the present invention and the pin or pins and the slot or slots may be associated with either of the support elements. Also, these figures are not to scale and are exaggerated for clarity.

FIGS. 2a and 2b show a preferred embodiment of a slotted multi-degree of freedom hinge element using one pin and one slot. Located at the end of the upper support element 8 is a pin 32 extending to the side. The pin is fitted onto an elongated slot 30 located at the end of the lower support element 6. This configuration may be particularly advantageous due its low profile.

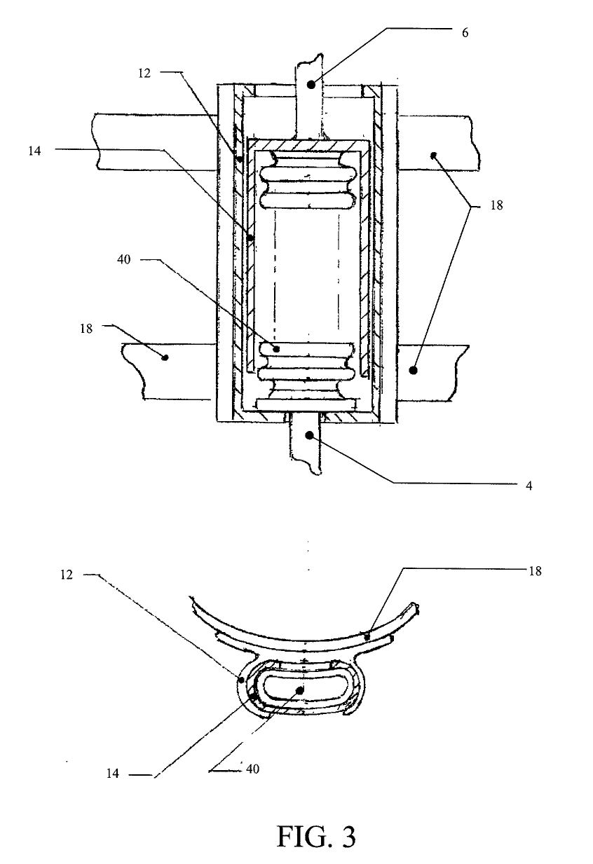

FIG. 3 shows an alternative preferred embodiment of a load-distance compensation device. The structural elements are similar to those described with regards to the load-distance compensator of FIG. 1 and are so numbered, with the exception of the pneumatic spring 40 that is shown here. It will be obvious to one skilled in the art, that further alternate load-distance compensators may include other forms of mechanical, pneumatic, electromechanical, electromagnetic, or hydraulic devices or systems.

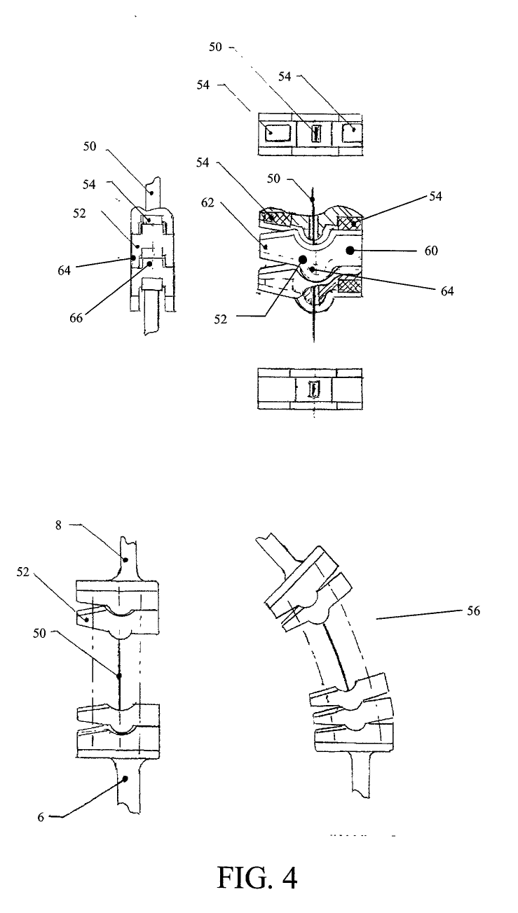

FIG. 4 provides a series of side, front, top, and bottom views showing an alternative preferred embodiment of a hinge element upon which the upper and lower support systems rotate. In the multi-degree of freedom hinge element shown here, the upper support element 8 and the lower support element 6 are fixed to opposite ends of a leaf spring 50 that is position so as to bend in a direction 56 compatible with the primary direction of rotation of the knee. The leaf spring is encased in a plurality of bend restriction links 52. The rectangular profile of the forward portion 60 of each of the links restricts the forward rotation of the hinge element. The angled profile of the rear portion 62 of each of the links allows for limited rotation within the normal bending limits of the knee. The juxtaposition of the lobes 64 and the raised central portion 66 of the adjacent link restricts the side movement and twisting of the links and the leaf spring. Because there are a number of bend restriction links, the leaf spring is free to bend substantially anywhere along its length. This freedom allows the axis about which the upper and lower support systems rotate to vary in location and orientation as needed to accommodate the natural bending of the knee. The cushions 54 serve to soften the impact of the links when one link impacts another as rotation is halted.

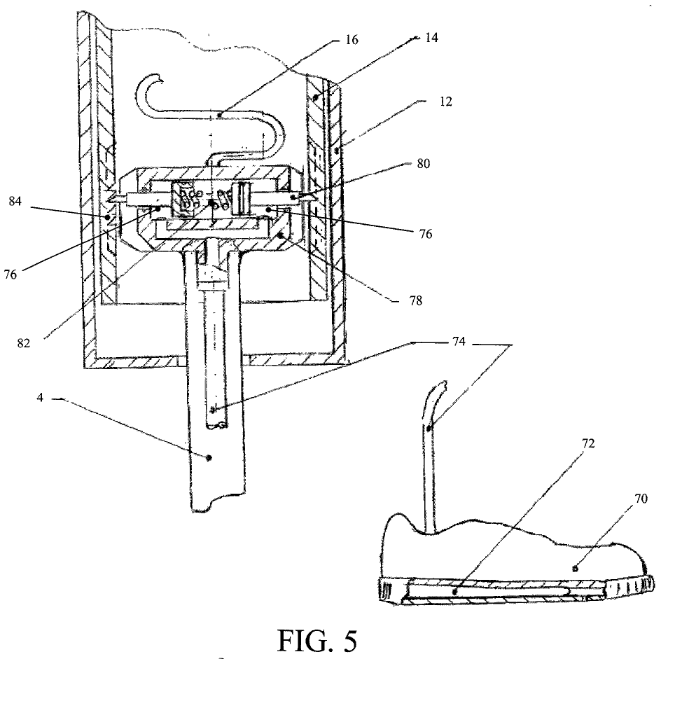

While the configuration of a control mechanism or associated trigger mechanism may vary, possible examples include, but are not limited to, piezoelectric actuators, pressure switches, electromagnetic solenoids, electromechanical devices, and fluidic systems. By definition fluids include gases and liquids so that fluidic system will include hydraulic and pneumatic systems. FIG. 5 shows a preferred embodiment of a control mechanism that uses hydraulic pressure to trigger the load-distance compensation device. The control mechanism selectively restricts expansion of the spring mechanism of the load-distance compensation device. This prevents the extension of the spring element of the load-distance compensator, and in this embodiment, prevents over extension of the surface contact extension when it is not in contact with a surface. When a person wearing the supplementary support brace takes a step, the foot applies pressure to the hydraulic fluid bladder 72 in the sole of the shoe 70. The hydraulic fluid is forced up the tube 25 that is located inside of, or adjacent to, the lower rigid support element 4 and connects the bladder to the control mechanism housing 78. As the hydraulic pressure in the chambers 76 increases, the locking pins 80 compress the retaining spring 82 and disengage from the locking notches 84. This allows the piston 14 to move freely as the spring 16 acts upon it, thus supplying the necessary force for the brace to take the load of the step rather than the knee. As pressure is taken off the bladder, the retaining spring forces the locking pins back into the locking notches. It should be noted that the use of a control mechanism with embodiments not employing a surface contact extension may be valuable and is within the intentions of the present invention.

It will be appreciated that the above descriptions are intended only to serve as examples, and that many other embodiments are possible within the spirit and the scope of the present invention.

What is claimed is:

1. A device that provides supplementary support to a joint so as to reduce the load applied to the joint by transferring a portion of the load through the device, the joint being located between a proximal body portion and a distal body portion, the device comprising:
(i) a proximal support system including at least one proximal support element, and at least one proximal attachment structure, said proximal attachment structure configured for attachment to portions of the proximal body portion and for transfer of the portion of the load between the proximal body portion and said proximal support system;
(ii) a distal support system including at least one distal support element, and at least one distal attachment structure, said distal attachment structure configured for attachment to portions of the distal body portion and for transfer of the portion of the load between the distal body portion and said distal support system; and
(iii) a load-transfer system interconnected with said proximal support system and said distal support system, said load-transfer system including:
( [symbol] ) a load-distance compensator configured so as to accommodate changes in the distance over which the portion of the load is transferred while supplying a force so as to cause said proximal support system and said distal support system to move substantially away from each other; associated with
( [symbol] ) at least one hinged element configured so as to allow rotation of said proximal and distal support systems in relation to each other so that said rotation accommodates natural bending of the joint.

2. The device of claim 1, wherein said proximal attachment structure includes at least one strap.

3. The device of claim 1, wherein said distal attachment structure includes at least one strap.

4. The device of claim 1, further comprising a control mechanism, said control mechanism selectively regulating said load-distance compensation device.

5. The device of claim 4, further comprising a trigger mechanism configured to actuate said control mechanism.

6. The device of claim 5, wherein said trigger mechanism includes a fluidic system.

7. The device of claim 1, wherein said load-distance compensator supplies a substantially constant force.

8. The device of claim 1, wherein said load-distance compensator includes a mechanical spring mechanism.

9. The device of claim 1, wherein said load-distance compensator includes a pneumatic spring mechanism.

10. The device of claim 1, wherein said load-distance compensator includes a combination of spring mechanisms.

11. The device of claim 1, wherein at least a portion of said load-distance compensator is incorporated proximal to said hinge.

12. The device of claim 1, wherein at least a portion of said load-distance compensator is incorporated distal to said hinge.

13. The device of claim 1, wherein said hinge e lement includes a member with one degree of translational freedom of movement, said movement being substantially perpendicular to a direction of said force.

14. The device of claim 13, wherein said hinge element includes a bracket associated with a first of said proximal and distal support elements, and further includes at least one pin associated with a second of said proximal and distal support systems, said bracket containing at least one elongated slot, said elongation being in a direction substantially perpendicular to said first support element and substantially parallel to a plane that is perpendicular to a primary axis of rotation of said knee, said pin being fitted into said slot, said pin being substantially parallel to said primary axis of rotation of said knee, said slot allowing for sliding movement and rotation of said pin.

15. The device of claim 1, wherein said hinge element includes a leaf spring fixedly attached to corresponding ends of said proximal support element and said distal support element, said spring configured so as to bend in a direction compatible with a primary direction of rotation of said joint, said spring being encased by a plurality of bend restriction links, said links configured so as to allow said spring to bend within rotational limits of said joint while restricting said bending outside of said rotational limits of said joint.

16. A device that provides supplementary support to a human leg so as to reduce the load applied to the knee while standing or walking by transferring a portion of the load through the device, the device comprising:
(i) an upper support system including at least one upper support element, and at least one upper attachment structure, said upper attachment structure configured for attachment around portions of the leg above the knee and for transfer of the portion of the load between the leg above the knee and said upper support system;
(ii) a lower support system including at least one lower support element, and at least one lower attachment structure, said lower attachment structure configured for attachment around portions of the leg below the knee and transfer of the portion of the load between the leg below the knee and said lower support system; and
(iii) a load-transfer system interconnected with said upper support system and said lower support system, said load-transfer system including:
( [symbol] ) a load-distance compensator configured so as to accommodate changes in the distance over which the portion of the load is transferred while supplying a force so as to cause said upper support system and said lower support system to move substantially away from each other; associated with
( [symbol] ) at least one hinged element configured so as to allow rotation of said upper and lower support systems in relation to each other so that said rotation accommodates bending of the knee.

17. The device of claim 16, wherein said lower support system is supported by, and transfers the portion of the load solely to, the leg below the knee.

18. The device of claim 16, further comprising a surface contact extension interconnected with said lower support element, said lower support element being supported by attachment to the leg and additionally supported by said surface contact extension coming in contact with a surface upon which a foot of said leg steps, the portion of the load being transferred to the leg and said surface.

19. The device of claim 18, wherein said lower support element is supported primarily by said surface contact extension and the portion of the load is applied primarily to said surface, said lower support element being attached to said leg primarily for positioning purposes.

20. The device of claim 18, further comprising a control mechanism, said control mechanism selectively restricting downward extension of said surface contact extension.

21. The device of claim 20, further comprising a trigger mechanism configured to actuate said control mechanism.

22. The device of claim 21, wherein said trigger mechanism includes a hydraulic system.

23. A device that provides supplementary support to a human leg so as to reduce the load applied to the knee by transferring a portion of the load to the device, the device comprising:
(i) an upper support system including at least one upper support element, and at least one upper attachment structure, said upper attachment structure configured for attachment around portions of the leg above the knee and for transfer of a portion of the load between the leg above the knee and said upper support system;
(ii) a lower support system including at least one lower support element that extends downward along the leg so as to contact a surface upon which a foot attached to the leg is resting, and at least one lower attachment structure, said lower attachment structure configured for attachment around portions of the leg below the knee and for transfer of a portion of the load between said surface and said lower support system; and
(iii) a load-transfer system interconnected with said upper support system and said lower support system, said load-transfer system including:
( [symbol] ) a load-distance compensator configured so as to accommodate changes in the distance over which the portion of the load is transferred while supplying a force so as to cause said upper support system and said lower support system to move substantially away from each other; associated with
( [symbol] ) at least one hinged element configured so as to allow rotation of said upper and lower support systems in relation to each other so that said rotation accommodates bending of the knee.

24. The device of claim 23, further comprising a support member associated with said upper support system, said support member configured for deployment adjacent to at least one force transfer surface of a bone structure inclined so as to be significantly non-parallel to a direction of force transfer, thereby facilitating relatively direct transfer of the portion of the load on to the upper support system.
